(19) United States
(12) Patent Application Publication
(10) Pub. No.: US 2013/0226065 A1
(43) Pub. Date: Aug. 29, 2013

(54) DIALYSATE PROFILING CONTROLLED BY UV MONITORING

(71) Applicants: Henrik Wolff, Witzenhausen (DE); Stefan Moll, Melsungen (DE)

(72) Inventors: Henrik Wolff, Witzenhausen (DE); Stefan Moll, Melsungen (DE)

(73) Assignee: B. Braun Avitum AG, Melsungen (DE)

(21) Appl. No.: 13/821,659

(22) PCT Filed: Sep. 29, 2011
(86) PCT No.: PCT/DE2011/001785

§ 371 Date: May 17, 2013

(30) Foreign Application Priority Data
Sep. 28, 2010 (DE) 10 2010 047 215.8

(51) Int. Cl. A61M 1/30

(52) U.S. Cl. CPC A61M 1/30
USPC 604/5.01

(57) ABSTRACT

The invention relates to a method and an apparatus for the determination of removed uremic substances from an extracorporeal blood circulation and for an adjustment of flow controlled according to this data. The method according to the invention is used for the optimization of the consumption as well as the use of a volume of dialysate.

### CROSS REFERENCE TO RELATED APPLICATIONS

This application is the U.S. national phase application of PCT International Application No. PCT/DE2011/001785 filed Sep. 29, 2011, which claims priority to; German Patent Application No. 10 2010 047 215.8 filed Sep. 29, 2010, the contents of each application being incorporated by reference herein.

### FIELD OF THE INVENTION

The invention relates to a method and an apparatus for the determination of removed uremic substances from an extracorporeal blood circulation and for an adjustment of flow controlled according to this data. The method according to the invention is used for the optimization of the consumption as well as the use of a volume of dialysate.

### BACKGROUND OF THE INVENTION

In patients with partial or complete renal failure waste products of the natural metabolism including uremic toxins are removed by means of blood treatment method, such as the hemodialysis, wherein the removal of the substances from the blood is carried out extracorporeal by the contact of the blood with a dialysis solution in the so-called extracorporeal blood circulation. The substance transport from the blood into the dialysis solution is carried out over diffusive and convective effects. The aim is that primarily uremic toxins are removed. This is done by adding the vital substances for the patients in physiological concentration of the dialysis solution.

The measure of the dialysis dose of a patient can not be carried out only based on the most subjective evaluation of the patient's health. It is necessary to quantify the dialysis success in a way so that an adequate dialysis performance is ensured. At the same time, a too high level of dialysis is to be avoided for cost reasons. To make the dialysis treatment more efficient, it is necessary to control the dialysis efficiency during the treatment in order to control this by adjusting the variable parameters of the blood treatment unit manually or automatically.

To ensure an adequate dialysis therapy, the Kt/V model was developed. Urea is the main metabolic end product in the blood to be purified. Therefore, urea is used for the determination of an adequate dialysis therapy. K is the clearance of the dialyzer of urea from the blood in ml/min. t is the treatment time in min and V is the urea distribution volume in ml in the human body, which is in direct relation to the weight of the patient. The dimensionless factor Kt/V is a factor for the reduction of nitrogen bound in urea in the blood of a patient. The temporal process is shown in FIG. 1. The determination of the urea concentration and/or of the concentration of other toxic substances in the dialysate outflow provides a comprehensive monitoring of dialysis progress. However, samples must be still now taken manually from the extracorporeal system, and be sent for the chemical analysis to a suitably equipped laboratory. Thus, a direct control of the dialysis machine is still excluded during the dialysis of each patient. Since patients requiring dialysis are often chronically ill and must undergo dialysis regularly, in such a control method an increased staffing need and analysis costs are steady incurred. Also the frequent sampling from the extracorporeal blood circulation results in a small but real risk of contamination with bacteria and viruses.

Therefore, a continuous monitoring of hemodialysis was required (IEEE Engineering in Medicine & Biology Society 11^{th} International Conference; Proceedings). A parameter known from the prior art is the change of the conductivity of the dialysis solution by the hydrolysis of urea and/or other important molecules. The calibration of conductivity sensors which are developed specially for this application, has proved in practice to be very tedious and unstable, since influences on the conductivity can also come from other sources. Moreover, by means of conductivity measurements none of single substances but only the entity of electrolytes is measured, which is quite inaccurate.

Furthermore, the continuous monitoring of a hemodialysis over optical absorption measurement can be performed. The transmission of the dialysis solution is influenced mainly from uric acid and other small molecule substances. Such a measuring device is described through the UV monitoring system by Fridolin in EP 1 083 948 B1.

In the EP 1 543 852 B1 a dialysis machine is described, in which the measurement of the hematocrit in the blood of the patient is also used to correct an ideal curve of the blood and treating parameters on the basis of this measurement. A measurement of the UV absorption is not provided in this method.

The quotient of the production of uric acid to urea is largely constant in patients, independent on the degree of the renal failure. In other words, the amounts of urea and uric acid formed per time unit correlate well with each other. The elimination rate of the both substances are under physiological conditions quite similar. Therefore, the concentration measurement of the uric acid in dialysate outflow is directly in connection with the flow of the dialysis solution a factor for the amount of removed urea. The advantage of measuring the absorption of uric acid is in the fact that uric acid has in contrast to urea a sharp and characteristic band in the UV range, which is between 280 nm and 290 nm.

### SUMMARY OF THE INVENTION

The objective of the invention is to provide an online monitoring system with which the flow of the dialysis solution and/or of the blood can be controlled. Yet, such a direct method for the optimization of the dialysis operation, and its implementation in a blood treatment unit is not available, because the data of the dialysis quality—if at all obtained—only be determined after the treatment, and thus an acute adjustment is not possible.

Surprisingly it was found that data obtained via a photometric concentration measurement system can be used not only for the analysis and monitoring the success of the dialysis procedure, but also for calculating the control of the blood treatment unit. In the present invention a method and an apparatus is described, in which the measured value obtained online can be used for the control of the entire dialysis procedure. This objective is achieved by the provision of methods and apparatuses according to the independent claims. Further advantageous embodiments, aspects and details of the invention are disclosed in the dependent claims, the figures and the description.

Basis of this invention is an online monitoring of the dialysis quality that determines by means of photometric absorbance in the dialysate outflow the dialysis performance in the form of the Kt/V value. In preferred embodiments, the UV absorbance is measured

In further embodiments the photometric absorbance can be measured alternatively or additionally in the blood flow. In preferred embodiments this blood-side measurement is carried out between patient access and dialyzer receipt. Thus, the following descriptions and embodiments are related not only to the dialysate-side, but also blood-side photometric absorbance.

Under the generic term of blood treatment unit all devices are understood which can be used for purification and/or treatment of blood. The most commonly used method are the double needle hemodialysis, single needle hemodialysis, single needle cross over hemodialysis, peritoneal dialysis, hemoperfusion, post-dilution hemodiafiltration, pre-dilution hemodiafiltration, pre-post-dilution hemodiafiltration, post-dilution hemofiltration, pre-dilution hemofiltration, pre-post-dilution hemofiltration or a sequential hemodialysis.

The term dialysis refers to purification methods in which two liquid streams are separated by a permeable membrane, but which enables the desired exchange of substances. The one liquid stream, in this case the blood, leads the substances to be removed with itself, while the other stream with the dialysis solution should take these substances.

An important and known dialysis method is the hemodialysis, which is performed as standard for the blood washing in a partial or complete renal failure. Herein, the blood to be purified is dialyzed extracorporeally via a semipermeable membrane against the dialysis solution. Uremic toxins are removed from the blood and the purified blood is returned to the body's blood circulation. To avoid that by the dialysis physiologically important substances are removed from the blood, the dialysis solution is enriched with the very same substances. So focusing on the removal of uremic toxins and waste products is achieved.

The underlying principle of the dialysis is the filtration, in particular the tangential flow filtration (TFF; also known as cross flow filtration). The blood to be purified is led through a filter module of hollow fibers, the actual dialyzer or dialysis filters. The wall of these hollow fibers is separated by a semipermeable membrane from the cleaning solution (dialysis solution). The dialysis solution has typically a lower concentration of the substances that are to be removed from the liquid to be purified. This difference in concentration leads to a diffusion. To make optimal use of the diffusion as separation force, tangential filters are nowadays preferably operated by the countercurrent principle.

Another purification mechanism is convection. Here a pressure gradient across the dialysis filter is produced, whereby the liquid to be purified is pressed reinforced through the semi-permeable membrane. Thus, the substances in its present concentration are purged along. In the hemodialysis, this effect is relatively small, since only the physiologically necessary liquid volume is removed from the patient. In the case of convective therapy forms such as hemofiltration and hemodiafiltration fluid is removed consistently from the blood via this mechanism, it is called ultrafiltration. This purification process is therefore not dependent on a concentration gradient of the substances concerned. Decisive here are membrane and material properties such as pore diameter, filtration distance, sieving coefficient, permeability etc. the sieving coefficient is a function of the molecular size, the electric charge, the shape, and the aggregate state of the substance to be removed. Permeability is the quotient from transported amount of substance per time and the product from the concentration gradient and the passage area.

The liquid volume removed by ultrafiltration should be substituted up to a natural amount of water to be eliminated, which corresponds to the natural urine volume. This can be done by substitution with a substitute solution, typically physiologic salt solution.

In addition to the dialysis therapies, there are other blood purification methods, such as apheresis, that suffer independent on renal failure from acute symptoms, such as hyperkalemia, metabolic acidosis, overhydration (eg, pulmonary oedema), uremic serositis (eg pericarditis or uremic encephalopathy) or poisoning with dialyzable substances such as lithium or acetyl salicylic acid. Chronically occurring symptoms that indicate a dialysis are, among others a low glomerular filtration rate, hyperphosphatemia or uremia. The inventive method for the control of the related treatment method can be used also for the treatment of the aforementioned symptoms or diseases. The blood purification methods further described are also often subsumed under the generic term dialysis.

A method for the continuous treatment of patient with renal failure is the peritoneal dialysis. Herein it is an intracorporeal method, in which the membrane properties of the peritoneum are used. The abdominal cavity of the patient is in this case filled with the dialysis solution, which is fed via two ports in and out.

In the hemoperfusion (e.g. in case of poisonings) the blood is led extracorporeal over an adsorbent such as charcoal or an exchange resin in order to remove the concerning toxins from the blood.

In the hemofiltration water is removed convectively from the blood. Here, an applied pressure gradient pushes the blood through a membrane. Thereby, uremic substances are also flushed out together. The volume loss is compensated by the addition of electrolyte solution.

In the method of the hemodiafiltration the classical hemodialysis and the hemofiltration are combined with each other in order to take the advantages of both methods. In this way molecules with low and medium molecular weight can be eliminated. Therefore, the hemodiafiltration is a preferred blood purification method in which the present invention can be applied.

The term ultrafiltration refers here to the filtration over a membrane with a pressure from 0 to 1 bar, in which particles bigger than about 0.01 μm should be retained. This corresponds to the size of medium molecular substances but also of macromolecules, viruses, and colloids.

With the demographic changes and the associated changes in the age pyramid of a society above all in industrialized countries these methods gain a steadily growing importance, as the number of patients with chronic renal failure increases continuously. Thus, there are at present, for example, in Germany about 60,000 patients requiring dialysis per year. The mortality rate is still at about 20% per year. The incidence of new cases is 184 per 1 million inhabitants. Also in the emerging economies, the number of patients to be treated will rise strongly in the coming years.

An average hemodialysis session lasts 4 to 5 hours. A night dialysis lasts up to 8 hours. Such a treatment is necessary in most patients at least three times a week. The treatment frequency depends inter alia on the body weight, the residual renal function and the cardiac output of the patient. The trend is, however to carry out rather several shorter treatments.

To cover this immense demand it requires an elaborate infrastructure, and along with that a large staff requirements. Also the material consumption is enormous. Thus, in an average 5-hour dialysis about 150 liters of dialysis solution pass through the dialyzer. Extrapolated, this means that a dialysis patient per year requires about 23,000 liters of dialysis solution. As due to the high quality requirements for dialysis solution, a bag is expensive despite the simple chemical composition, the treatment costs of a dialysis patient per year are to about £43,000. For 60,000 patients, this would be for Germany alone approximately £2.58 billion. This is a significant part of the public health budget. Therefore, it is of general interest, to achieve the saving potentials during dialysis treatments most extensively.

In these enormous costs, it seems necessary to realize saving possibilities, wherein the quality of medical treatment but may not suffer. This means that the uremic substances still need to be removed in a medically acceptable percentage from the patient's blood. To ensure this, the concentration of the lead compounds to be removed must be determined reliably.

With the inventive method an optimization of the dialysis efficiency is intended. The dialysis efficiency results from the interaction of the costs used in the dialysis and the dialysis performance. For the dialysis performance an own measure must be found. In the scientific literature, a number of calculations have been established to express the dialysis performance. Some of the values are very similar, while others put a different emphasis on the consideration of the problem. According to the invention the method is able to provide measured values for all of the established parameters, and combinations thereof, so that they can be considered in the control of the dialysis process.

The most commonly used factor for a dialysis performance parameter is the so-called Kt/V-model. Urea is the important metabolic end product in the blood to be purified. Thus the urea concentration is an excellent parameter, with which the performance of an adequate dialysis therapy can be better understood. K is the clearance of the dialyzer of urea from the blood in ml/min, t is the treatment time in min and V is the urea distribution volume in ml in the human body, which is in direct relation to the weight of the patient. The dimensionless factor Kt/V represents the reduction of urea-nitrogen in the blood of a patient.

In the hemodialysis Kt/V values ≧1.2 are intended. In a process of a dialysis without technical problem such a value can be achieved as standard.

According to Daugirdas formula:

$$K \cdot \frac{t}{V} = - ln [ \frac{{C}_{t}}{{C}_{0}} - 0.008 \cdot t + ( 4 - 3.5 \frac{{C}_{t}}{{C}_{0}} ) \cdot \frac{UF}{W} ]$$

Herein means:
Cₜ urea concentration at the end of the dialysis
C₀ urea concentration at the beginning of the dialysis
t dialysis time in hours
UF ultrafiltration volumes in liter
K clearance in ml/min
W dry weight in kilogram.
   (cited in: Halwachs-Baumann: Labormedizin: Klinik—Praxis—Fallbeispiele. 2. Ed. Springer Wien New York, 2011, S. 298).

A simple factor is the URR (urea reduction ratio)

URR [%]=(C₀−Cₜ)/C₀*100

C₀ is the concentration of urea at the time 0 (beginning of the dialysis or the treatment cycle) Cₜ is the concentration of urea at actual time t. URR [%] is the percentage that was eliminated at time t. Values are intended to be greater than 65%.
sp Kt/V (single-pool Kt/V) takes into account both the urea production during the dialysis and the effect of the ultrafiltration:

spKt/V=−ln(R−0.008*t)+(4−3.5*R)*UF/W

Herein R=Cₜ/C₀, t is the dialysis time in hours, UF is the ultrafiltration volumes in L and W is the weight after the hemodialysis in kg.

The eKt/V value (equilibrated Kt/V) also takes into account the urea rebound that occurs even after the end of dialysis. The urea rebound refers to the effect that after the end of dialysis the urea concentration in the blood rises again relatively quickly, because now the urea existing in low circulated tissues and not caught by the dialysis passes over more and more into the whole organism.

eKt/V=spKt/V−0.6/t·spKt/V+0.03

This formula applies to a dialysis through a peripheral shunt.

TAC/TAD is a factor, how evenly the urea concentration in the blood displays over a total period. This is particularly interesting in the aspect, whether the selected dialysis regime and the associated dialysis dose produce the desired success. Herein, TAC is the average weekly urea concentration (time average concentration). TAD refers to the fluctuation of the TAC values, and is thus a derived factor. Small TAD values are intended, because this is a sign that dangerous and potentially toxic peak values in the urea concentration occur rarely or not at all.

EKR describes the urea clearance. Herein is

EKR=G/TAC.

G is here the formation rate of urea.
RU refers to the amount of the removed urea.
SRI however refers to solute removal index and considers the dialysis performance of the reverse side:

SRI=1−(Vₚₒₛₜ*Cₚₒₛₜ)/(Vₚᵣₑ*Cₚᵣₑ)

(Vₚₒₛₜ: volume after the dialysis; Cₚₒₛₜ: concentration after the dialysis;
Vₚᵣₑ: volume before the dialysis; Cₚᵣₑ: concentration before the dialysis)
V refers to the urea distribution volume in blood (see above: Kt/V)
K is a factor for the clearance performance of a specific dialyzer. It corresponds to the effective body clearance.

All these aforementioned parameters can be summarized in the sense of the invention under the term “dialysis performance parameter(s)”.

According to the invention, in analogous manner measured values for the purification of further important uremic substances can be obtained. Therefore, the figures and embodiments made in the description are equally applicable to these substances. In addition to urea and uric acid these can be among other things creatinine and hippuric acid.

The measured values obtained according to the invention can be retrieved with a previously created profile for this specific machine and/or patients. This profile can provide, for example, required output values. Thereby, the treatment can begin early with machine configurations close to optimal dialysis efficiency.

For an optimized clearance it is especially in the case of the diffusive substance transport meaningful to adapt the flow of the dialysis solution to the treatment conditions or to get information, how the treatment can be optimized from an economical point of view. The relation between dialysate flow and clearance is shown in FIG. 2. The optimization is carried out mainly according to economical criteria. Therapeutic aspects may optionally be also considered. These may be included in the pre-selection of the operating mode and a lowest acceptable value for a dialysis performance parameter, but have then no influence any more on the optimization according to economical criteria.

An economization, i.e. a saving of the cost-intensive dialysis solution in the required quantities can then be achieved if it is recognized that a reduction of the flow of the dialysis solution does not result in the reduction of the clearance. This may be the case if for example a large-area filter is coupled with a high dialysate flow. In this case, it may happen that the blood has, for example, in the half way through the filter already a concentration of uremic toxins, which tends toward very low values. In this case, the flow of the dialysis solution can be reduced without causing a deterioration of the clearance. The only change resides in that the blood is not almost completely purified half way through the filter, but only toward the end of the filtration distance. Therefore the treatment can be optimized in such a way that the potential of the filter is fully used in order to keep the consumption of dialysis solution as low as possible.

It is specifically pointed out that the inventive method is related exclusively to a method for the optimization of the consumption of dialysate. The changes of the configuration based on this method in the dialysis machine have no influence on the diagnosis of the patient, the treatment type, the treatment efficiency and/or the success of treatment. The inventive method is only for economic considerations for the intelligent economical use of the dialysis machine. This has for the patient itself no positive or negative effect.

To date there is no application of the detected photometric signal for the control of the dialysis process, since data on the dialysis quality and related process—if at all applicable—are determined only ex posteriori and therefore timely intervention is not possible. Also from signals of the conductivity in the dialysis solution, such a control is not known.

In the opposite case, it may happen that an increase of the dialysate flow causes an increase of the clearance. This is the case when the blood leaves the dialyzer, however, has still a relatively high concentration of uremic substances. By an increase of the flow of the dialysis solution the concentration gradient in the filter can be increased and thus the clearance can also be improved. The aim is to produce the optimum flow of the dialysis solution for the current situation.

An adjustment of the flow of dialysis solution may be performed either in predefined or freely selectable intervals or due to abnormalities in the online signal of the purification monitoring. If for example, the signal remains over specific time period mainly unchanged and/or displays very high absolute values, it is assumed that the filter is demanded to perform over its performance potential. Accordingly, the flow of dialysis solution must be increased.

In the opposite case, at very low values of online monitoring is the possibility to decrease the flow of the dialysis solution.

In preferred embodiments, the each saved amount of dialysis solution is displayed as an absolute value and/or based on a time interval.

There is also the possibility to define at the beginning of treatment, the volume of dialysis solution, which is to be expended during the treatment and ensures the desired clearance. This value can be achieved either by a constant flow over the therapy, or also by intelligent staggered profiles. In contrast to a constant flow, a profile which begins with high flow rates, which decrease over the treatment process, has the advantage that at the beginning of the therapy the basis for a good dialysis therapy is established and the loss of quality is kept low, if complications occur toward the end of treatment.

Further, the invention relates to a method in which the flow of the dialysis solution is varied during a dialysis session. Via such a method, measured values are obtained which, when they are related to one another, give an indication whether the flow of blood and/or the dialysis solution must be readjusted or in the sense of economy can be readjusted. For the necessity of the readjustment, a tolerance interval can be defined, within which the deviations of an expected value are tolerated for the respective time, and by exceeding the tolerance interval, however, must be readjusted. The expected value results from an extrapolation of the first measured values during a dialysis session. In addition, empirical values for the each building type of dialyzer, for the individual dialyzer and from the patient history can be considered for the expected value. For the variation of the flow of the dialysis solution a minimum value and a maximum value are determined, between which the whole variation range of the flow of dialysis solution is varied. This can be carried out according to a predetermined scheme, or input of a user.

Deviations of the limits of tolerance intervals and/or of the interpolated dialysis course can be used as an opportunity to make another variation of the flow of dialysis solution.

In addition, a time period can be determined, within which the variation is carried out. Also, the change rate can be determined with which the variation is performed. This variation can be carried out several times during a dialysis session. Also, this variation can take place at defined intervals, or individually as required. In preferred embodiments, the time intervals at the beginning of a dialysis session are shorter than at the end, as usually toward the end a smaller variability of the measurement result occurs. For this method the same embodiments and modifications described in the present invention are applied.

According to the invention the flow of the dialysis solution can also be varied, if required. This is particularly the case, if the photospectroscopic measured values have abnormalities, which indicate evidence to an unexpected and/or incomplete process of the dialysis session.

Such an peculiar course can consist of a very slow decline or a very large value of the removed substance amount. As an action of remedy, an increase in the flow of dialysis solution would be proposed or made.

Conversely, the peculiar course can consist of a very small value of the removed substance amount. In this case a decrease in the flow of the dialysis solution would be proposed or made.

If any one of the described abnormalities shall happened, this can also be checked in the case of the dialysate volume selected at the beginning of the treatment and adjusted if necessary, as described above.

In summary, the invention can be described as follows:

During a dialysis treatment by means of an online measurement method the concentration of at least one removed substance in the dialysate is monitored and on the basis of this signal the flow of the dialysis solution is so adjusted that an optimal saturation of the dialysis solution with respect to uremic substances is reached.

In the case of the determination of the volume of dialysate, this course is used at the beginning of the therapy in order to determine the volume of dialysate that is expended in the therapy.

To determine the optimal flow the dialysate flow is varied and the generated absorbance change is analyzed.

The above-described effect can also be generated by adjusting the flow of the fluid to be purified, the blood. Crucial factors are often not the absolute flows of individual compartments, but the relative flow of the two relative to each other.

The sampling rate for the photospectroscopic signal can be either preset by the manufacturer or be input by the users. According to the invention the sample and control intervals are between 0.5 seconds and 30 minutes, preferably between 1 second and 10 minutes, more preferably between 2 seconds and 5 minutes, and most preferably between 10 seconds and 1 minute.

In some embodiments, however, the measuring intervals can be extended during a dialysis treatment, either according to a predetermined algorithm, or according to individual configurations. Background to this is that the treatment parameters change according to experience more strongly at the beginning of a dialysis treatment than toward the end, at which a “quasi-steady state” is usually reached.

In further embodiments, firstly at least two measuring points are recorded. These are used for the characterization of the clearance. Another curve is interpolated. Herein, the measuring points with a constant frequency can be recorded or the measuring intervals can be optimized on the expected course.

Preferred are photospectroscopic methods. Herein, the measuring beam is irradiated perpendicularly to the flow direction of the dialysis solution. An IR measuring beam is already used in many modern dialysis machines. It is used for the detection of so-called blood leaks in the semipermeable membrane of the dialysis module. If here a leak occurs, unfiltered blood goes into the dialysis solution, and thereby is withdrawn from the recirculation into the body of the patient. Depending on the extent of the leak, this can be fatal for a patient. The leaked blood can be detected by turbidity in the dialysis solution and then the dialysis session can be interrupted or disrupted. The problem in measuring aqueous solutions, however, are two very broad characteristic bands for H₂O, which overlap many measuring values. IR measurements are therefore only of interest to the concentration measurements, in which the lead compound has at least one characteristic band outside both H₂O bands.

Polarized light can be used only with lead compounds that have firstly a chiral center, and if they do so are provided as a racemate. Since urea is not chiral, such a method cannot be used for urea measurement. With other lead substances that fulfill this condition, the concentration c would be calculated as follows:

c=α/(αₜ·d)

(α=measured rotation angle; αₜ=specific rotation ability of a substance; d=measuring distance, here the diameter of the dialysate outflow; α and αₜ are wavelength-specific)

Particularly preferred is therefore the application of at least one UV absorbance measuring unit. Herein, a UV light beam is passed though a segment of the solution to be measured and is measured by a receiver. From this measured value the extinction or as the reciprocal value, the transmission can be calculated at a specific wavelength in the UV range (1-400 nm wavelength). The extinction is linear to the concentration for a wide concentration range for most compounds. This is generally described by the Beer-Lambert law:

E_{λ}=ε_{λ}·c·d

The extinction E_{λ} is the product from the substance-specific molar extinction coefficient ε_{λ}, the substance concentration c and the distance of the light measured d.

Since ε_{λ} and d are determined for an apparatus, E_{λ} is a direct measure for the substance concentration to be measured.

The UV measurement is highly temperature dependent, because the temperature directly affects the extinction coefficient ε_{λ}. Therefore, in preferred embodiments, precautions are made such that the temperature of the dialysis solution is maintained constant at least in the dialysate outflow in order to ensure comparability of the measured values.

For the UV absorbance measurements, commercially available sensors can be used. In preferred model systems the fluid stream flows through a transparent outflow component such as a tube which is guided through a central recess on the UV sensor. In other words, the UV sensor surrounds the outflow component in a section in ring-shaped manner. The UV beam passes herein through the central segment of the outflow component in its full width. This leads to an increase of the measurement accuracy, as in this way scattering and diffraction effects are reduced. In the selection of the wall material of the outflow component transparent at least in this section, it is advantageous if materials are used, in which only a low level of light scattering and reflection occurs. These two effects affect also negatively the measurement accuracy. Especially for measurements below 200 nm wavelength quartz glass has been proven useful. According to the invention, this glass may be also incorporated in the sensor and the dialysis solution flows on the route by the sensor freely through this glass tube. Producing corresponding connections before and after the sensor is within the knowledge of the average person skilled in this field. The error accuracy of the measurement system should be preferably less than 15%, more preferably less than 10% and most preferably less than 5%.

For reasons of protection, if necessary, due to the design, screens should be attached to the measuring area of the UV sensor to prevent that human tissue, especially the eyes of the staff and/or the patient comes into contact with the UV measuring beam.

The concentration measurement of the substance or substances to be removed can be made according to the invention at all points in the dialysis compartment. But it is preferred that the measurement is made at a point with the maximum concentration of the substance to be removed in the dialysis solution. This serves to obtain the most reliable measured values of the respective concentration. This is usually the case in the outflow component on the dialysis side. Particularly preferred is the measurement at the entrance of the outflow component, since during the passing through of the fluid through the outflow component the concentration is not further increased, but in the further course already diffusion effects can occur within the outflow component, which can affect the measurement.

For the connection of the outflow component to the dialysis filter, it has proved to be advantageous that there are no hard edges or abrupt narrowing of the flow path. If possible a laminar flow profile, as it usually is present in the dialysis filter shall remain. Otherwise at edges and constrictions turbulences can occur, which can lead at these points to the unequal distribution of the transported substances and thus potentially to imprecisely measured values. Since these turbulences are depending on the adjustable flow rate and it can be assumed that these concentration changes can be non-linear, turbulences can be a error source. Commercially available dialysis tubes meet this criterion.

As adjustable factors to a blood treatment unit at hemodialysis machine especially the flow of the blood and the flow of the dialysis solution are important. Further, for example, the ultrafiltration rate through the balance chamber, or the applied transmembrane pressure, the treatment duration of the patient can be adjusted and the speed (the switching interval of the balancing chamber) and the amount of volume compensation can be controlled.

Since the hemodiafiltration is a combination of hemodialysis and hemofiltration, the same parameters are adjustable as configurable in the individual procedures.

The method according to the invention can be carried out in principle to all mammals. Preferably, however, is the use in the treatment of human patients.

Critical physiological factors which hinder the unlimited controllability of the dialysis course are blood flow and ultrafiltration rate.

Therefore, in preferred embodiments, the hematocrit and/or the plasma protein concentration are determined by suitable additive processes and can be incorporated into the calculation of the optimal dialysis machine configurations as limiting factors (for example, for the ultrafiltration rate). According to the invention a calibration can be take place with the previously stored literature values or it can be stored individually for each dialysis machine type, a single machine and patient values of one or more dialyses as comparison values and be used for evaluation.

More frequently occurring problems are hypotensive episodes in patients with a corresponding vulnerability. This can lead to a circulatory failure. Therefore in some dialysis machines, the blood pressure is controlled automatically. In some machines, an alarm is triggered when falling below a critical value. Usually upon this as a reaction interruption of the ultrafiltration or a reduction of the blood flow is enforced. In very critical cases the flow of dialysis solution is stopped. Due to the lack of diffusion the clearance is extremely reduced.

If it is known by the patient history, that at a correspondingly high combination of blood flow and dialysis solution flow hypotensive episodes have occurred, the thus well-known critical values form an upper limit, below which the regulation of the two flows takes place. If the analysis of the photospectroscopic measurement in this case should recommend flows above the known critical values, it is provided that the flows remain below the critical values upon inclusion of the patient history.

Natural limits of controllability of the two flows in the hemodialysis are given therefore on the one hand by the technical capabilities of the respective dialyzer as well as material properties, for example, dialysis tubes. On the other hand, too high rates of blood flow can also have a negative effect. Also on the blood side turbulences can occur and shearing forces take effect. The physiologically possible extracorporeal blood flows are however usually low enough so that no large shearing forces occur.

The two flows for the blood Q_{B} and the dialysis solution Q_{D} can be set in a quotient Q_{D}/Q_{B} to each other.

According to the invention three different modes of the dialysis efficiency can be defined depending on the quotient Q_{D}/Q_{B}:

### a) Eco Mode: 0.5<Q_{D}/Q_{B}<1.3

### b) Efficiency Mode: 1.3≧Q_{D}/Q_{B}≧2.0

### c) Power Mode: 4.0>Q_{D}/Q_{B}>2.0

or expressed by the reciprocal value:

### a) Eco Mode: 2.0>Q_{B}/Q_{D}>0.77

### b) Efficiency Mode: 0.5≦Q_{B}/Q_{D}≦0.77

### c) Power Mode: 0.25<Q_{B}/Q_{D}<0.5

The same also applies to the volumes used over the therapy, which were determined at the beginning of a dialysis session. The ratio between blood volume V_{B} and volume of dialysate V_{D} can therefore be used in other embodiments.

### a) Eco Mode: 0.5<V_{D}/V_{B}<1.3

### b) Efficiency Mode: 1.3≦V_{D}/V_{B}≦2.0

### c) Power Mode: 4.0>V_{D}/V_{B}>2.0

or expressed by the reciprocal value:

### a) Eco Mode: 2.0>V_{B}/V_{D}>0.77

### b) Efficiency Mode: 0.5≦V_{B}/V_{D}≦0.77

### c) Power Mode: 0.25<V_{B}/V_{D}<0.5

Since the ranges given to the respective volumes-based modes are identical to those of the flows Q_{B} and Q_{B}, there is no distinction in the further description of the application modes. According to the invention, the term Application Modes thus relates to both classification criteria for the modes.

The Eco Mode (Economy Mode) is characterized in that Q_{B} (V_{B}) is not significantly larger than Q_{D} (V_{D}). This means that relatively little dialysis solution is used in order to archive a qualitatively still good dialysis result. A considerable amount of dialysis solution as well as its disposal can be saved. If the focus of the dialysis is on a possible economical operation, the Eco Mode is especially preferred.

The Efficiency Mode forms a middle range of the quotient Q_{D}/Q_{B} (V_{D}/V_{B}). In this range, the best compromise between efficiency and optimal dialysis result is sought. For the majority of applications, this mode will be particularly preferred.

The Power Mode is characterized by a particularly high flow of the dialysis solution in comparison to the blood flow. In this mode, an optimal or near-optimal clearance is achived. However, for this a disproportionate amount of dialysis solution is consumed. This mode is particularly preferable if due to abnormal high measured values for uric acid (and thus indirectly for urea), caused by acute renal failure (for example, in multi-organ failure or after surgery) or in acute poisoning, it is medically indicated to achieve as soon as possible a very extensive purification of the blood. The cost issue is here of less importance.

It should be noted that according to the invention also further operating modes based on the ratio Q_{D}/Q_{B} (V_{D}/V_{B}) as well as the respective reciprocal value can be determined and can be combined with each other. The boundary values of the Q_{D}/Q_{B} (V_{D}/V_{B}) and the respective reciprocal value and the designation of the individual operating modes can therefore be varied according to the invention.

According to the invention it can be specified by the user at a central processing unit, in which mode a control should preferably take place. The central processing unit checks whether the current incoming measured values and the resulting adjustments of the dialysis machine with the stored values for this control mode should be matched. In the positive case, an adjustment of the dialysis machine, especially of the flows of the blood and the dialysis solution is performed within the planned range for this mode for the quotient Q_{D}/Q_{B} or the reciprocal value. In the negative case the central processing unit displays a proposal, to which mode the dialysis machine should be switched. Optionally, an automatic conversion can also be performed.

The central processing unit can be any machine that has a CPU, a display device and an input device, for example a computer. The central processing unit can be attached to the dialysis machine itself or can be operated independently thereof. The same applies to a display device for the measured measurement values. Displaying these values can be done via the display device of the central processing unit or via an own display device. The transmission of the photospectroscopic measured values can be done via all devices known in the prior art, for example via a cable connection or a WLAN connection.

The course of the clearance can be compared with a model course saved at the central processing unit and a corresponding corrective action can be proposed or made.

For each patient, the measured values and flows determined during his dialysis sessions can be recorded on a data storage medium and made available for reuse.

In other embodiments the construction-related dialyzer specifications can also be used for the optimization of the blood purification process. Herein, the patient-specific data, and the dialyzer specifications can also be combined.

The collected data can be used for the generation of flow profiles, which are in turn proposed to the user.

On the display device, the current optimization process can be displayed to the user. This data can be saved on a data storage medium. In the same way, the saved amount of dialysis solution and/or the increased dialysis quality can be displayed.

Accordingly, the method for the optimization of the consumption of dialysate in a blood treatment unit according to the present invention comprises the following steps:
a) Preselection of an operating mode and a lowest acceptable value for a dialysis performance parameter;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the selected dialysis performance parameter; or
e′) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the selected dialysis performance parameter;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

The steps are executed in the given sequence from a) to g). In the above mentioned method, the steps e) and e′) are alternatives, i.e. the inventive method comprises the steps a) to g) or a) to d), e′), f) and g).

The present invention thus refers among other things to the following methods. Method for the optimization of the consumption of dialysate in a blood treatment unit, the following steps:
a) Preselection of an operating mode and a lowest acceptable value for a dialysis performance parameter;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the selected dialysis performance parameter;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session. a)

If the volume of the dialysis solution should be varied, the method according to the invention is as follows:
a) Preselection of an operating mode and a lowest acceptable value for a dialysis performance parameter;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e′) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the selected dialysis performance parameter;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

If a specific available volume of dialysate is given, there is the following method according to the invention:
a) Preselection of an operating mode and of a lowest acceptable value for a dialysis performance parameter;
a′) Preselection of a volume of dialysate to be consumed in the dialysis session;
b) Determining the current measured values for the flow of the blood and the dialysate in the blood treatment unit;
c) Measuring the UV absorbance of at least one uremic substance in the dialysate outflow or in the extracorporeal blood circulation of the blood treatment unit; Forwarding the measured values of the UV absorbance and the current measured values for the flow of the blood and the dialysate to a central processing unit;
e″) Determining a flow optimized on the selected dialysis performance parameter or an optimized sequence scheme of the flows of the dialysis solution, and/or the blood and/or a relative ratio of both flows within a Q_{D}/Q_{B} or V_{D}/V_{B} range determined for the selected operating mode in the central processing unit in compliance with the volume of dialysate to be consumed in the dialysis session;
f) Displaying the determined change of the flows of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

The step a′) is added in comparison with the aforementioned method and step e″) occurs instead of step e) or step e′), wherein step e″) includes an alternative with respect to the Q_{D}/Q_{B} range and the V_{D}/V_{B} range. The two methods which are covered by this alternative comprise the following steps: a), a′), b) d), followed by step e):
e) Determining a flow of the dialysis solution optimized on the selected dialysis performance parameter or an optimized sequence scheme of the flows of the dialysis solution and/or the blood and/or a relative ratio of the two flows within a Q_{D}/Q_{B} range determined for the selected operating mode in the central processing unit in compliance with the volume of dialysate to be consumed in the dialysis session;
   and steps f) and g).

The alternative method comprises the following steps:

a), a′), b)-d), followed by step e′):
e′) Determining a flow of the dialysis solution optimized on the selected dialysis performance parameter or an optimized sequence scheme of the flows of the dialysis solution and/or the blood and/or a relative ratio of the two flows within a V_{D}/V_{B} range determined for the selected operating mode in the central processing unit in compliance with the volume of dialysate to be consumed in the dialysis session;
   and steps f) and g).

If the flow of the dialysis solution should be varied, the method according to the invention is as follows:
a) Preselection of an operating mode and a lowest acceptable value for a dialysis performance parameter;
b) Determining the current measured values for the flows of the blood and the dialysate in the blood treatment unit;
c) Variation of the flows of the dialysis solution in the blood treatment unit during a dialysis session;
d) Measuring the UV absorbance of at least one uremic substance in the dialysate outflow or in the extracorporeal blood circulation of the blood treatment unit;
e) Forwarding the measured values of the UV absorbance and the current measured values for the flow of the blood and the dialysate to a central processing unit;
f) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the selected dialysis performance parameter;
g) Displaying the determined change of the flows of the dialysis solution and/or of the blood at the central processing unit and/or automatic readjustment in accordance with the calculated change in the blood treatment unit; and
h) Repeating the steps b) to f) after a respective predetermined time interval or time interval scheme until the end of dialysis session.

If in the abovementioned embodiment the flow of the dialysis solution should be varied, the method according to the invention is as follows:
a) Preselection of an operating mode and a lowest acceptable value for a dialysis performance parameter;
b) Determining the current measured values for the flows of the blood and the dialysate in the blood treatment unit;
c) Variation of the flows of the dialysis solution in the blood treatment unit during a dialysis session;
d) Measuring the UV absorbance of at least one uremic substance in the dialysate outflow or in the extracorporeal blood circulation of the blood treatment unit;
e) Forwarding the measured values of the UV absorbance and the current measured values for the flow of the blood and the dialysate to a central processing unit;
f) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the selected dialysis performance parameter;
g) Displaying the determined change of the flow of the dialysis solution and/or of the blood at the central processing unit and/or automatic readjustment in accordance with the calculated change in the blood treatment unit; and
h) Repeating the steps b) to g) after a respective predetermined time interval or time interval scheme until the end of dialysis session

The method for optimizing the use of a volume of dialysate in a blood treatment unit comprises according to the invention the following steps:
a) Preselection of an operating mode and a lowest acceptable value for a dialysis performance parameter;
a′) Preselection of a volume of dialysate to be consumed in the dialysis session;
b) Determining the current measured values for the flow of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e″) Determining an optimized sequence scheme of the flows optimized on the selected dialysis performance parameter of the dialysis solution, and/or the blood and/or a relative ratio of both flows within a Q_{D}/Q_{B} or V_{D}/V_{B} range determined for the selected operating mode in the central processing unit in compliance with the volume of dialysate to be consumed in the dialysis session;
f) Displaying the determined change of the flows of the dialysis solution and/or of the blood at the central processing unit and/or automatic readjustment in accordance with the calculated change in the blood treatment unit; and
g) Repeating the steps b) to f) after a respective predetermined time interval the time interval scheme until the end of dialysis session.

If in the abovementioned embodiment the flow of the dialysis solution should be varied, the inventive method for optimizing the use of a volume of dialysate in a blood treatment unit is as follows:
a) Preselection of an operating mode and a lowest acceptable value for a dialysis performance parameter;
a′) Preselection of a volume of dialysate to be consumed in the dialysis session;
b) Determining the current measured values for the flow of the blood and the dialysate in the blood treatment unit;
c) Variation of the flows of the dialysis solution in the blood treatment unit during a dialysis session;
d) Measuring the UV absorbance of at least one uremic substance in the dialysate outflow or in the extracorporeal blood circulation of the blood treatment unit;
e) Forwarding the measured values of the UV absorbance and the current measured values for the flow of the blood and the dialysate to a central processing unit;
f) Determining an optimized sequence scheme of the flows optimized on the selected dialysis performance parameter of the dialysis solution, and/or the blood and/or a relative ratio of both flows within a Q_{D}/Q_{B} or V_{D}/V_{B} range determined for the selected operating mode in the central processing unit in compliance with the volume of dialysate to be consumed in the dialysis session;
g) Displaying the determined change of the flows of the dialysis solution and/or of the blood at the central processing unit and/or automatic readjustment in accordance with the calculated change in the blood treatment unit; and
h) Repeating the steps b) to g) after a respective predetermined time interval or time interval scheme until the end of dialysis session.

As preferred dialysis performance parameters the Kt/V can be used. As preferred uremic substance uric acid can be used.

The inventive method in use of uric acid and Kt/V is as follows:
a) Preselection of an operating mode and a lowest acceptable value for the Kt/V;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the Kt/V;
e′) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the Kt/V;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval, or the time interval scheme until the end of dialysis session

Both the composition and the viscosity of the blood of the patient, the selected type of the dialysis machine, and its condition as well as especially the current state of the dialyzer membrane contribute to the diversity and unpredictability of the conditions to be determined and the resulting optimization options and proposals for change. Therefore, it is intuitively obvious that the optimization options and proposals for change can not result from a master formula, but must be determined individually for each case. For this it is referred back to the empirical method. Corresponding values can be determined from the previous process of the dialysis session, previous dialysis sessions for each patient in this dialysis machine, the patient history and the dialyzer specifications. An important role plays the above-described variation of the flow of dialysate during a dialysis session. The more empirical values are available, the more reliable the dialysate consumption can be optimized by a suitable adjustment of the flow of dialysis solution, and/or the blood and/or a relative ratio of the two flows. Analogously in this wayan optimized sequence scheme of the flows of the dialysis solution, and/or the blood and/or a relative ratio of the two flows can be determined to each other.

Similarly, the present invention relates to apparatuses which are suitable to implement the above-described inventive method. In particular, the present invention relates therefore to an apparatus for the blood treatment comprising
a blood treatment unit,
a UV measuring device and
a central processing unit,
   wherein the central processing unit has means which calculates a selected dialysis performance parameter according to the method of the invention for optimization of the use of dialysate in the blood treatment unit from the value determined of the values of the UV absorbance in dialysate outflow measured by the UV measuring device and determines on the basis of this a configuration of the flows of the blood and the dialysate optimized on the consumption in the dialysis solution, and has means to display this optimized configuration and/or to implement it automatically to the blood treatment unit.

In other words, the inventive apparatus for the blood treatment comprises a blood treatment unit, a UV measuring device and a central processing unit, wherein the central processing unit has means which can implement a method according to any one of claims 1-19, such as an apparatus for the blood treatment comprising
a blood treatment unit,
a UV measuring device and
a central processing unit,
   wherein the central processing unit has means which can implement a method comprising the following steps:
a) Preselection of an operating mode and a lowest acceptable value for a dialysis performance parameter;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the selected dialysis performance parameter; or
e′) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the selected dialysis performance parameter;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

Similarly, the present invention relates to an apparatus for the blood treatment comprising
a blood treatment unit,
a UV measuring device and
a central processing unit,
   wherein the central processing unit has means which calculates a selected dialysis performance parameter according to the method of the invention for optimization of the use of a predetermined volume of dialysate in the blood treatment unit from the value determined of the values of the UV absorbance in dialysate outflow measured by the UV measuring device and determines on the basis of this a configuration of the flows of the blood and the dialysate optimized on the consumption in the dialysis solution, and has means to display this optimized configuration and/or to implement it automatically to the blood treatment unit.

In other words, the inventive apparatus for the blood treatment comprises a blood treatment unit, a UV measuring device and a central processing unit, wherein the central processing unit has means which can implement a method according to claim 2.

Said means which calculates a selected dialysis performance parameter according to the method of the invention for optimization of the use of a predetermined volume of dialysate in the blood treatment unit and/or the inventive method for optimizing the use of a predetermined volume of dialysate in a blood treatment unit from the value determined of the values of the UV absorbance in dialysate outflow measured by the UV measuring device and determines on the basis of this a configuration of the flows of the blood and the dialysate optimized on the consumption in the dialysis solution, as well as the means display this optimized configuration and/or implement it automatically to the blood treatment unit, refer to hardware components of the central processing unit (usually a PC) known from the prior art as well as software specifically made for this purpose.

The terms “construction-related dialyzer specifications” or “dialyzer specifications” refer in the present invention to the technical details and connections that result from the production-related design of each dialysis machine and cannot be changed by the functional use of the dialysis machine. They provide the framework within which configurations can be made for the functioning of the dialysis machine, either manually, automatically, or through a computer-aided control.

The term “patient-specific data” in the sense of the invention refers to information relating to specific data of the patient for diagnosis, other diseases, medication, anamnesis and previous documented dialysis treatment. This data is individual, but may be part of a treatment profile for similar cases.

Under the term “flow” in the sense of the invention, the physical flow is understood, which represents the volume for fluids (and gases) which moves per time unit through a given cross-section of a flow carrier. Or it is represented as a formula:

$$Q = \dot{V} = \frac{d V}{d t}$$

Herein Q is the flow, \dot{V} is the volume flow rate, V is the volume and t is the time.

The term “flow profile” refers according to the invention to a scheme of the time sequence of combinations of the flows of blood and dialysate, or the dialysate alone. Herein, at least one of the flows and/or the time sequence can be predetermined or result from the calculation of the one or more dialysis performance parameters. Preferably, the determined or predetermined flow profiles are stored on the central processing unit and can be retrieved from there by the user. Such a stored flow profile can be used as an initial, but variable, or as the definitive basis of the sequence of a dialysis session. Analogous to the term “flow profile” the term “sequence scheme of the flows” can also be used in the sense of the invention.

The term “lowest acceptable value for a dialysis performance parameter” describes in the sense of the invention the limit value of the selected dialysis performance parameter, below which the dialysis performance should not fall at any time of the dialysis session. This limit value may be exceeded. A farthest possible excess is not medically necessary and often not economically meaningful. The extent of the limit value is different, of course, depending on the selected dialysis performance parameter. The selection and the extent are subject to individual patient-specific medical aspects (as the treatment purpose and diagnosis) and the performance criteria of the used dialysis machine and of the selected dialysis method. There is no mutual preconditioning between the selection of the dialysis performance parameter and its extent on the one hand and the maximum saving of the dialysate on the other hand.

In FIG. 1, the absorbance of a UV measuring signal in dependence on the duration of a dialysis session is depicted. This results in an exponential decay. This corresponds to the observation that at the beginning of a dialysis session, a relatively high measured value for the concentration of the substance to be removed from the blood, for example uric acid, is found. With increasing purification also the concentration of the removed substance in the dialysis solution decreases and strives asymptotically towards a minimum value. This minimum value is a correlate of the maximum clearance. As the initial value (100%) is a determined start value is used. For this purpose, the first measured values are determined. A first measured value may be typically obtained after about 7 minutes of treatment time. From these measured values a starting value can be back-calculated by extrapolation. Since reasonably in the first minutes of a dialysis session purification of the blood cannot yet be measured, the extrapolation is performed based on a fictitious beginning of the clearance, which is several minutes after the beginning of the dialysis session. This value is used as the initial value. For technical measurement reasons the theoretically expected sigmoidal beginning of the curve cannot be represented empirical.

In FIG. 1 it is shown on the basis of the absorbance that the concentration of absorbent substances in the dialysate falls rapidly or toward the end of the therapy the curve flattens very strong, which is caused less by the deterioration of the dialyzer's state than by a much lower concentration in the blood of the patient.

FIG. 2 shows a further characteristic of the treatment. The clearance of the dialyzer is shown in dependence on the dialysate flow. Without dialysate flow no purification of the blood can be performed, which corresponds to a purification of 0%. If the dialysate flow is increased, the percentage of clearance correspondingly increases. A 100% clearance would mean in this representation that 100% of the blood flowing into the dialyzer leave the dialyzer purified.

Other factors such as initial concentration, blood flow and filter properties can influence this process additionally.

### BRIEF DESCRIPTION OF THE DRAWINGS

FIG. 1: Curve of the absorbance of a UV measuring signal in dependence on the treating time.

FIG. 2: Curve of the percentage of clearance in dependence on the flow of the dialysis solution.

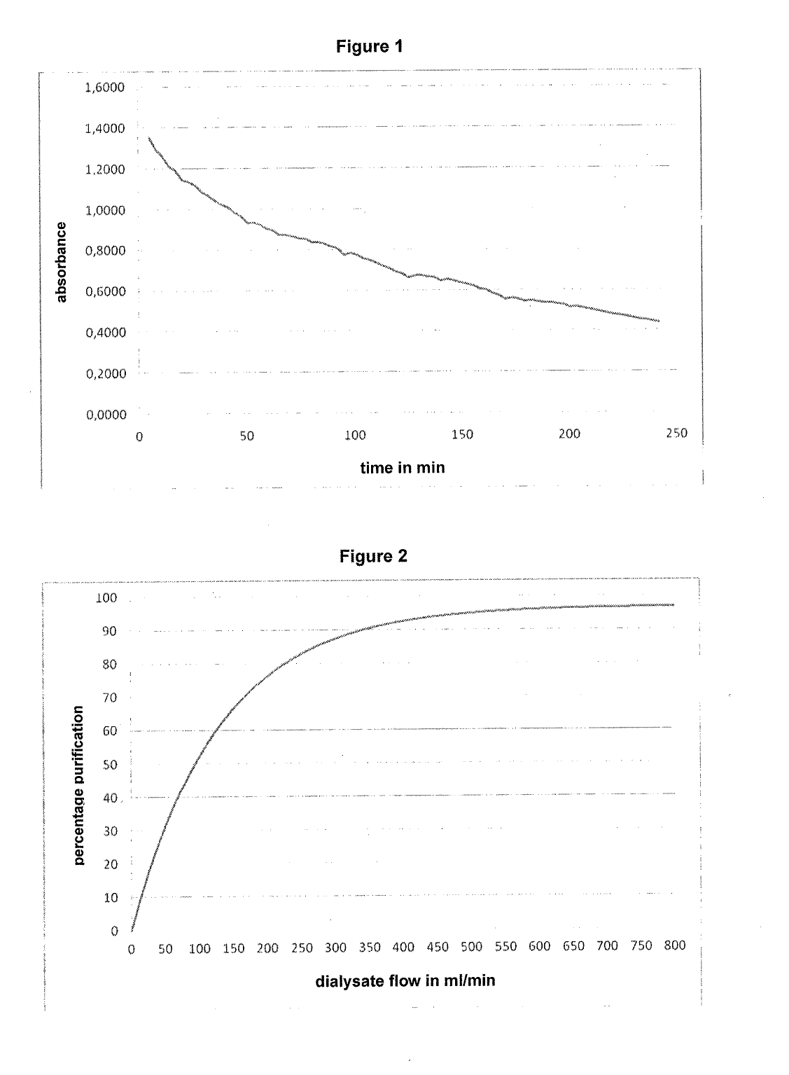

### EXAMPLES

### Example 1

For the determination of the following values it is assumed that after 2 hours of a dialysis session at a dialysate flow of 500 ml/min the flow of dialysate is to be reduced in order to achieve an optimal saving of dialysate available under these circumstances. For this, the flow of dialysate is reduced to 400 ml/min and in a parallel experiment to 300 ml/min and the dialysis was let run in this manner for additional 2 hours.

This resulted in the following values:

| BF | Kt/V(norm) | Kt/V(500, 400) | Kt/V(500, 300) |
|---|---|---|---|
| 400 | 2.16 | 2.07 | 1.95 |
| 300 | 1.68 | 1.635 | 1.56 |
| 200 | 1.17 | 1.155 | 1.125 |
| saved Volume | 0 | 12 | 24 |

Herein, BF represents the blood flow in ml/min; Kt/V(norm) is the measured value for the dialysis performance parameter Kt/V at a dialysate flow of 500 ml/min (an acknowledged standard value), Kt/V (500,400) is the Kt/V at a reduction of a dialysate flow of 500 ml/min to 400 ml/min and Kt/V (500,300) is the Kt/V at a reduction of a dialysate flow of 500 ml/min to 300 ml/min.

In the bottom line, the saved volume of dialysate over the time period of 2 hours is given in liters.

It is shown that a corresponding reduction of the dialysate flow results in only a small deterioration of the Kt/V. If the lowest acceptable value f or Kt/V is appropriately selected, or the standard configurations for the first phase of the dialysis session is selected with sufficient tolerance, the low deteriorations of Kt/V remains above the lowest acceptable value for Kt/V, and there is no loss in the predetermined dialysis performance for the patient.

However, significant amounts of dialysate can be archived by such an inventive flow control, which is not available without the inventive adjustment of the flows (cf. 0 liter savings without adjustment).

1. Method for the optimization of the consumption of dialysate in a blood treatment unit comprising the following steps:
a) Preselection of an operating mode and a lowest acceptable value for a dialysis performance parameter;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the selected dialysis performance parameter; or
e′) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the selected dialysis performance parameter;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

2. Method according to claim 1 comprising the following steps:
a) Preselection of an operating mode and of a lowest acceptable value for a dialysis performance parameter;
a′) Preselection of a volume of dialysate to be consumed during the dialysis session;
b) Determining the current measured values for the flow of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e″) Determining a flow optimized on the selected dialysis performance parameter or an optimized sequence scheme of the flows of the dialysis solution, and/or the blood and/or a relative ratio of both flows within a Q_{D}/Q_{B} range or V_{D}/V_{B} range determined for the selected operating mode in the central processing unit in compliance with the volume of dialysate to be consumed during the dialysis session;
f) Displaying the determined change of the flows of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

3. Method according to claim 1, wherein said operating mode is selected from the group consisting of Eco Mode, Efficiency Mode and Power Mode.

4. Method according to claim 1, wherein the dialysis performance parameter is Kt/V.

5. Method according to claim 1, wherein for peculiar measured values the output of the results comprises the recommendation to change the operating mode or the automatic change of the operating mode.

6. Method according to claim 1, wherein before the measurement of the UV absorbance the following step is carried out:
Variation of the flow of the dialysis solution in the blood treatment unit during a dialysis session.

7. Method according to claim 6, wherein in predefined or freely selectable time intervals the flow of the dialysis solution is varied.

8. Method according to claim 7, wherein the predefined time intervals over the process of the treatment remain constant.

9. Method according to claim 7, wherein the predefined time intervals become longer toward the end of the treatment.

10. Method according to claim 1, wherein in a peculiar course of the signal of the online monitoring the flow of the dialysis solution is varied.

11. Method according to claim 10, wherein the peculiar course is a very slow decline or a very large value of the removed substance amount, upon which as a consequence an increase of the flow of the dialysis solution is proposed or made.

12. Method according to claim 10, wherein the peculiar course is a very low value of the removed substance amount, upon which as a consequence a decrease of the flow of the dialysis solution is proposed or made.

13. Method according to claim 1, wherein the course of the clearance is compared with a model course saved on the central processing unit, and a corresponding corrective action is proposed or made.

14. Method according to claim 1, wherein the course of the clearance is defined by recording of individual measuring points and a curve is interpolated, wherein the measuring points are recorded with a constant frequency or optimized on the expected process.

15. Method according to claim 1, wherein measured values and flows determined for each patient during his dialysis sessions are recorded on a data storage medium and are made available for reuse.

16. Method according to claim 1, wherein construction-related dialyzer specifications are used for the optimization of the consumption or the use of dialysate.

17. Method according to claim 16, wherein patient-specific data and the dialyzer specifications are combined.

18. Method according to claim 15, wherein the collected data for the generation of flow profiles is used, which are proposed to the user.

19. Method according to claim 1, wherein the current optimization process is displayed to the user and is saved on a data storage medium.

20. Method according to claim 19, wherein the saved amount of dialysis solution or the increased dialysis quality is displayed.

21. Apparatus for the blood treatment comprising
a blood treatment unit,
a UV measuring device and
a central processing unit,
wherein the central processing unit has means which calculate a selected dialysis performance parameter according to the method from claim 1 from the value determined of the values of the UV absorbance in dialysate outflow measured by the UV measuring device and determines on the basis of this a configuration of the flows of the blood and the dialysate optimized on the consumption in the dialysis solution, and has means to display this optimized configuration and/or to implement it automatically to the blood treatment unit.

22. Apparatus for the blood treatment comprising
a blood treatment unit,
a UV measuring device and
a central processing unit,
wherein the central processing unit has means which can implement a method comprising the following steps:
a) Preselection of an operating mode and a lowest acceptable value for a dialysis performance parameter;
b) Determining the current measured values for the flows of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e) Determining a flow of the dialysis solution and/or the blood optimized on the consumption of dialysate, and/or a relative ratio of the two flows within a Q_{D}/Q_{B} range determined for the selected operating mode in the central processing unit without falling as a consequence below the lowest acceptable value of the selected dialysis performance parameter; or
e′) Determining a flow of the dialysis solution and/or the blood optimized on the consumption of dialysate, and/or a relative ratio of the volumes throughput of the dialysate and of the blood within a V_{D}/V_{B} range determined for the selected operating mode in the central processing unit without falling as a consequence below the lowest acceptable value of the selected dialysis performance parameter;
f) Displaying the determined change of the flow of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

23. Apparatus for the blood treatment comprising
a blood treatment unit,
a UV measuring device and
a central processing unit,
wherein the central processing unit has means which calculates a selected dialysis performance parameter according to the method of claim 3 from the value determined of the values of the UV absorbance in dialysate outflow measured by the UV measuring device and determines on the basis of this a configuration of the flows of the blood and the dialysate optimized on the consumption in the dialysis solution, and has means to display this optimized configuration and/or to implement it automatically to the blood treatment unit.

24. Apparatus for the blood treatment comprising
a blood treatment unit,
a UV measuring device and
a central processing unit,
wherein the central processing unit has means which can implement a method comprising the following steps:
a) Preselection of an operating mode and of a lowest acceptable value for a dialysis performance parameter;
a′) Preselection of a volume of dialysate to be consumed in the dialysis session;
b) Determining the current measured values for the flow of the blood and the dialysate in the blood treatment unit;
c) Measuring the UV absorbance of at least one uremic substance in the dialysate outflow or in the extracorporeal blood circulation of the blood treatment unit;
d) Forwarding the measured values of the UV absorbance and the current measured values for the flow of the blood and the dialysate to a central processing unit;
e″) Determining a flow optimized on the selected dialysis performance parameter or an optimized sequence scheme of the flows of the dialysis solution, and/or the blood and/or a relative ratio of both flows within a Q_{D}/Q_{B} or V_{D}/V_{B} range determined for the selected operating mode in the central processing unit in compliance with the volume of dialysate to be consumed in the dialysis session;
f) Displaying the determined change of the flows of the dialysis solution and/or of the blood at the central processing unit and/or automatic readjustment in accordance with the calculated change in the blood treatment unit; and
g) Repeating the steps b) to f) after a respective predetermined time interval or time interval scheme until the end of dialysis session.

25. Method according to claim 2, wherein said operating mode is selected from the group consisting of Eco Mode, Efficiency Mode and Power Mode.

26. Method according to claim 2, wherein the dialysis performance parameter is Kt/V.

27. Method according to claim 2, wherein for peculiar measured values the output of the results comprises the recommendation to change the operating mode or the automatic change of the operating mode.

28. Method according to claim 2, wherein before the measurement of the UV absorbance the following step is carried out:
Variation of the flow of the dialysis solution in the blood treatment unit during a dialysis session.

29. Method according to claim 2, wherein in a peculiar course of the signal of the online monitoring the flow of the dialysis solution is varied.

30. Method according to claim 29, wherein the peculiar course is a very slow decline or a very large value of the removed substance amount, upon which as a consequence an increase of the flow of the dialysis solution is proposed or made.

31. Method according to claim 29, wherein the peculiar course is a very low value of the removed substance amount, upon which as a consequence a decrease of the flow of the dialysis solution is proposed or made.

32. Method according to claim 2, wherein the course of the clearance is compared with a model course saved on the central processing unit, and a corresponding corrective action is proposed or made.

33. Method according to claim 2, wherein the course of the clearance is defined by recording of individual measuring points and a curve is interpolated, wherein the measuring points are recorded with a constant frequency or optimized on the expected process.

34. Method according to claim 2, wherein measured values and flows determined for each patient during his dialysis sessions are recorded on a data storage medium and are made available for reuse.

35. Method according to claim 2, wherein construction-related dialyzer specifications are used for the optimization of the consumption or the use of dialysate.

36. Method according to claim 35, wherein patient-specific data and the dialyzer specifications are combined.

37. Method according to claim 34, wherein the collected data for the generation of flow profiles is used, which are proposed to the user.

38. Method according to claim 2, wherein the current optimization process is displayed to the user and is saved on a data storage medium.

39. Method according to claim 38, wherein the saved amount of dialysis solution or the increased dialysis quality is displayed.

40. Method according to claim 6, wherein in predefined or freely selectable time intervals the flow of the dialysis solution is varied.

41. Method according to claim 40, wherein the predefined time intervals over the process of the treatment remain constant.

42. Method according to claim 40, wherein the predefined time intervals become longer toward the end of the treatment.

43. Apparatus for the blood treatment comprising
a blood treatment unit,
a UV measuring device and
a central processing unit,
wherein the central processing unit has means which calculate a selected dialysis performance parameter according to the method from claim 2 from the value determined of the values of the UV absorbance in dialysate outflow measured by the UV measuring device and determines on the basis of this a configuration of the flows of the blood and the dialysate optimized on the consumption in the dialysis solution, and has means to display this optimized configuration and/or to implement it automatically to the blood treatment unit.
